(19) United States
(12) Patent Application Publication
(10) Pub. No.: US 2018/0348981 A1
(43) Pub. Date: Dec. 6, 2018

(54) Method for Making and Using a Simplified Visual-Motor Evaluation and Training Device with an Improved User Interface

(71) Applicants: Richard Bonar Devies, Sandy, UT (US); Christopher Allen Davies, Sandy, UT (US)

(72) Inventors: Richard Bonar Devies, Sandy, UT (US); Christopher Allen Davies, Sandy, UT (US)

(21) Appl. No.: 15/609,884

(22) Filed: May 31, 2017

(51) Int. Cl. G06F 3/0488; H04N 5/33; G09B 19/00

(52) U.S. Cl. CPC G06F 3/0488; G09B 19/003; H04N 5/33

(57) ABSTRACT

Use of the herein disclosed novel Surface Projected Optical Touch Detection Systems (SPOT) user interface for a Visual Motor and Neuro-Cognitive Evaluation and Reaction Training Device (VMTD) eliminates all costs and potential maintenance issues associated with making the many wire connections between the illuminated electrical switch elements of the user interface portion of the VMTD device and connection of the user interface unit to the associated computer based system controller and other associated devices that comprise the complete VMTD.

### BACKGROUND OF THE INVENTION

The present invention relates, in general, to the operation of visual-motor and neuro-cognitive evaluation and sport training devices for use in evidence based medical rehabilitation and sport performance evaluation and the methods of making and using such devices and, more particularly, to a method to make and use a VMTD with an improved user interface that is simpler, lower cost and more reliable than user interface methods that are presently used in VMTD devices. Further, it will be well understood by the reader that the disclosed methods and teachings are suitable for effective use of such a user interface device in medical rehabilitation and sport training devices, including use of the disclosed methods in the design and function of devices used in a hospital or clinic setting, user's home, sport facility and military or industrial environments. This invention also has applications to improve the user interface portion of other electronic devices where the user interacts with the device by pressing an illuminated button or switch.

There are a number of visual-motor and neuro-cognitive evaluation and training devices being used in medically directed rehabilitation programs for persons recovering from stroke, traumatic brain injury (TBI), sport concussion, neuro surgery and other conditions or events affecting visual-motor and/or neuro-cognitive performance. Such devices are also widely used by athletes, sport teams and athletic trainers in the visual-motor and neuro-cognitive evaluation and training of athletes and by military personnel for tactical training.

By far, the most widely used device of this type is the Dynavision Visual-Motor Reaction Device; manufactured and distributed by Dynavision International, LLC, in Westchester Ohio. The Dynavision model 2000™ was first introduced into use in about 1990 and the follow-on model, the Dynavision D2™ replaced the model 2000™ in 2010. There are more than 1,600 Dynavision devices in use throughout the U.S. and in more than 30 foreign countries.

The Dynavision device is comprised of a large 4′×4′ board with a plurality of lighted mechanical button switches mounted on the face of the Board. The large face size of the board and the raised buttons are critical to its function—to challenge the entire visual field of the user and provide tactile feedback as the user stands (or sits) in front of the device and presses (or hits) the raised switches as they randomly light-up. When an illuminated button is pressed, that light turns off and another turns on. The device keep score of how fast and accurately the user can respond to the lights.

In addition to the plurality of lights (64 lights) on the face of the Dynavision™ board, there is a visual display located in the general center of the Dynavision board. Changing letters, words, numbers, shapes, etc. can be programmed to display on this central screen; and the user is instructed to react to this information (i.e., call out information on the screen, read the words, perform the calculation), while continuing to hit/press the lights as they illuminate. This is done to provide a measurable and repeatable neuro-cognitive challenge to the user and require the user to coordinate use of their central and peripheral vision capabilities.

There are other ‘large faced’ visual-motor reaction devices that are somewhat similar to the Dynavision device, such as the Vision Coach® (Perceptual Testing, Inc.) and BITS™ (Bioness, Inc.) devices. While both of these devices do have a large size face area populated with individual points or dots of light that challenge a wide visual field, similar to the Dynavision device, the Vision Coach® achieves the lighted targets by randomly illuminating the face of the board behind membrane switches that do not substantially protrude from the face of the device; as do the illuminated mechanical push button switches used in the Dynavision. Further, the Vision Coach™ does not have an information screen in the middle of the board.

Both the Dynavision™ and Vision Coach™ Devices are purpose designed and built for their intended use, such that, once designed, the entire board and electronics of each device must be separately fabricated, including installing multiple wires to each of the many illuminated, low profile button areas on the board and wiring to connect all of the lights on the user interface board portion of the device to the power source and to the computer based control portion of the device.

By comparison, the Bioness BITS™ device is created using a large commercially available, flat screen, touch activated computer monitor. The BITS™ system computer is programmed to create light ‘spots’ on the screen that the user is instructed to ‘touch’ to turn off the light, as another lighted ‘spot’ turns on.

Further to current devices, while the BITS™ device and its use of a commercially available touch screen activated computer monitor as the user interface eliminates the need for a significant number of parts, wiring and related manufacturing costs, when related to the Dynavision™ & Vision Coach™ and others; the BITS™ and Vision Coach™ devices, with their flat button design, totally loose the important benefits to the user of raised 3 dimensional lighted targets as well as eliminating the tactical feed-back experienced when the user touches a simply lighted dot on the flat screen. Raised, lighted, 3 dimensional targets are of particular importance when evaluating and training a user's peripheral vision.

In addition, and of great consequence, the user must touch or press the BITS' glass touch screen with some care. A hard strike to the screen with a fist, heal of the hand or knuckle can damage the monitor; and such strikes are common occurrences when the Dynavision™ is used by athletes pressing to improve their performance scores on such a training device. Further, some touch screen monitors will not respond to a touch by an object, e.g., a ball being held by a user of the BITS™ type device; as these common touch screens require contact with a part of the user's body to cause a change in the electrical capacitance being sensed at the location where the screen is being touched by a user's hand/finger.

### BRIEF SUMMARY OF THE INVENTION

This invention teaches how to make and use a visual-motor and neuro-cognitive reaction measuring and training device (VMTD) that completely eliminates the very significant manufacturing costs and potential maintenance issues associated with wiring and attaching by mechanical clips or soldering each of the many wires to and from each lighted switch and the need to replace failed switches, while retaining the important and very necessary features of user tactile feedback, illuminated and raised 3D buttons that can be struck repeatedly with a hard force or by an object, as well as by a finger or hand. The teachings of this invention also eliminate the need for and costs of installing a separate ‘Dynavision like’ LED or LCD information screen in the face of light board, a previously described.

This disclosure relates to the field of useful devices intended to measure, evaluate, train and improve human performance, including visual-motor reaction time and neuro-cognitive processing. More specifically, it relates to a method for making and using a simplified visual-motor and neuro-cognitive evaluation and reaction training device (VMTD) where the user interface portion of the device has no need for electrical wiring components or connections and no wireless communication capabilities between the user interface portion of the device and the computer controlling the VMTD and other element of this VMTD device, and such improved user interface results from the herein disclosed improvements to currently available Surface Projected Optical Touch Detection Systems (SPOT).

Examples of well understood and currently practiced Surface Projected Optical Touch Detection Systems (SPOT) include infrared (IR) rear-projection touch systems; IR Curtain touch system and a frustrated total internal reflection system; as noted and described per V. Sharma, Texas Instrument Inc., application U.S. Ser. No. 13/828,736, Sep. 19, 2013.

While each of the above referenced SPOT systems have various specific features that enable the function of devices that can create many of the functional elements of a computer touch screen when such image is projected on a wall or screen, none of the present SPOT systems are suitable for use as a user interface for a neuro-cognitive evaluation and visual motor reaction training device (VMTD) unless modified per the teachings herein, thereby creating a new and novel means of user interface for such VMTD devices.

Use of the herein disclosed novel Surface Projected Optical Touch Detection Systems (SPOT) user interface for a VMTD eliminates all costs and potential maintenance issues associated with making the many wire connections between the illuminated electrical switch elements of the user interface portion of the VMTD device and connection of the user interface unit to the associated computer based system controller and other associated devices that comprise the complete VMTD.

### DETAILED DESCRIPTION OF THE INVENTION

This invention is comprised of the following elements:

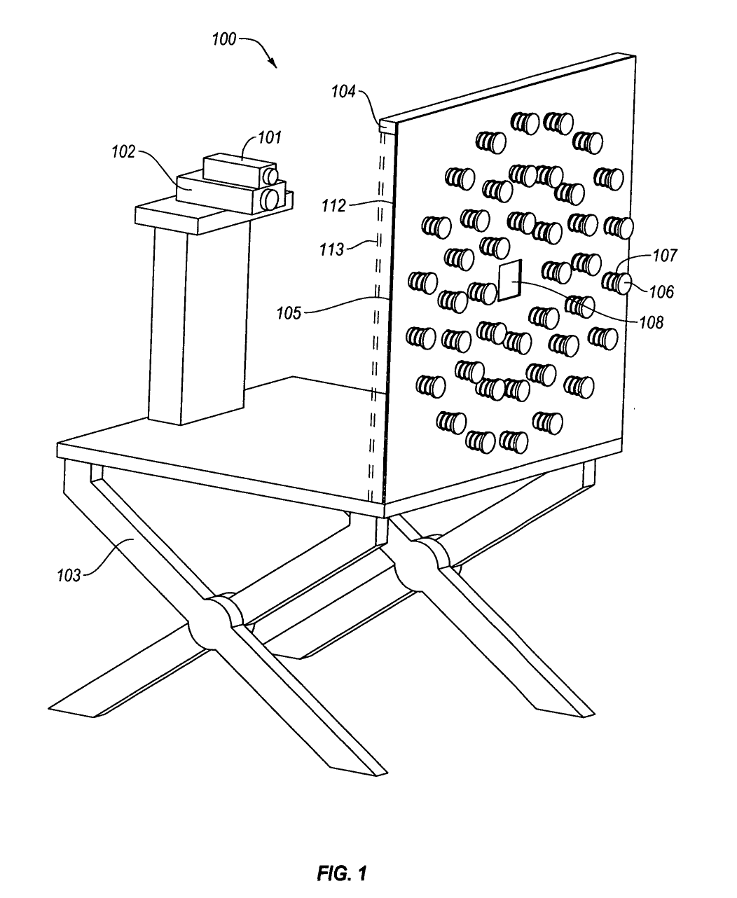

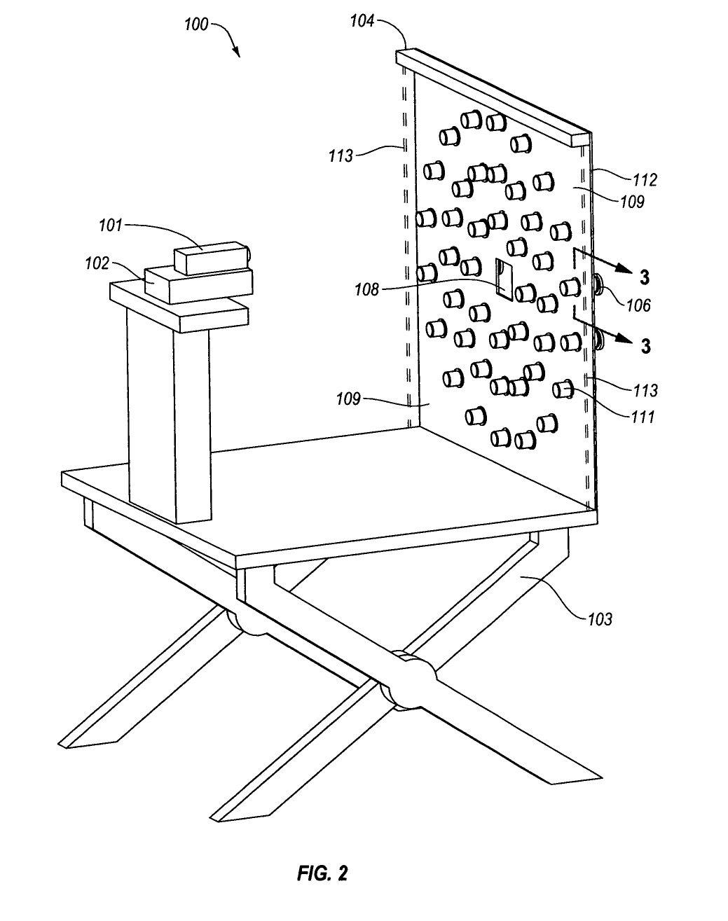

(a) USER INTERFACE BOARD (FIGS. 1, 2 & 3; Element 112) portion of the device that, in a preferred design, is a generally flat and square shaped board, fabricated of a flat, ridged, opaque material with a surface about 50 inches×50 inches with a front surface ‘User Side’ and a back surface ‘Rear Side’ and where the User Interface Board has one or more openings, about 0.5 inches in cross section, through the USER INTERFACE BOARD to provide for the installation of buttons into the board and an additional approximate 4 inch×5 inch translucent area in the general center of the USER INTERFACE BOARD that will display images that are visible on the User Side of the board when projected on the Rear Side of such translucent area. For ease of use, this USER INTERFACE BOARD may be mounted on a moveable stand that allows the board to be rolled from place to place and raised or lowered such that the general center of the Board is about the same height as the eye level of the user;
(b) BUTTON (FIGS. 1, & 2; Elements 106 & 111) or plurality of three dimensional BUTTONS that are fabricated of light transmitting clear plastic and that are secured to the User Side of the USER INTERFACE BOARD such that depression of a BUTTON by the user, from the User Side of the USER INTERFACE BOARD, causes a portion of the BUTTON to extend through and beyond the rear surface of the USER INTERFACE BOARD; (See FIG. 01)
(c) SYSTEM CONTROLLER comprised of a common and commercially available laptop computer running a standard PC operating system such as Microsoft ‘Windows 10’, Ubi Interactive Basic Touch Kit (Ubi Interactive, Seattle, Wash.) or equal software and custom operating software that controls the PROJECTOR's illumination of the BUTTONS, times and records the user's reaction time in responding to the illumination of a button and also causes the PROJECTOR to displayed information images on the USER INTERFACE BOARD's central display and; where no portion of the SYSTEM CONTROLLER is electronically connected by conductive wire or wireless radio signal to any portion of the USER INTERFACE BOARD and wherein all information transfer and communication between the said SYSTEM CONTROLLER and said USER INTERFACE BOARD is by way of light energy;
(d) PROJECTOR (FIG. 100, 200; Element 102) portion of the device such as a Canon LV WX 300 ST or similar, positioned to project visible light images onto the Rear Side of the USER INTERFACE BOARD, when such light images are created and controlled by the SYSTEM CONTROLLER, and in so doing selectively illuminates one or more of the clear plastic BUTTONS on the User Side of the USER INTERFACE BOARD by projecting light onto the portion of the clear plastic BUTTON that is visible from the Rear Side of the USER INTERFACE BOARD. There may also be a clear or translucent area of the USER INTERFACE BOARD located immediately surrounding a target BUTTON that is also illuminated by visible light from the PROJECTOR, to further highlight and illuminate the selected BUTTON to be addressed by the user;
(e) Infrared Emitter (FIGS. 1 & 2; Element 104) portion of the device as per Ubi Interactive, Seattle, Wash. or similar, that generates a broad infrared (IR) beam or curtain of IR light and that is mounted above and parallel to Rear Side of the USER INTERFACE BOARD such that the emitted IR curtain is parallel to the Rear Side of the USER INTERFACE BOARD and;
(f) Infrared Camera (FIGS. 1 & 2; Element 101) portion of the device as per Ubi Interactive, Seattle, Wash. or similar, mounted immediately above or below the PROJECTOR and directed at the Rear Side of the USER INTERFACE BOARD. This IR camera is connected to and controlled by the SYSTEM CONTROLLER.

In one preferred design a suitable BUTTON is fabricated from a clear, light transmitting Lucite plastic rod with a diameter or cross section of about 0.5 inches and a length of about 2.0 inches. Lucite or a similar clear plastic material is desirable due to its well understood ability to transmit visible light through its length and out its distal end, with up to 90% of the light that is projected onto the proximal end of the structure being transmitted to the distal end, (proximal being the end closest to the projected light source and on the Rear Side of the USER INTERFACE BOARD and the distal end of the BUTTON being the end furthest from the light source and being on the User Side of the USER INTERFACE BOARD).

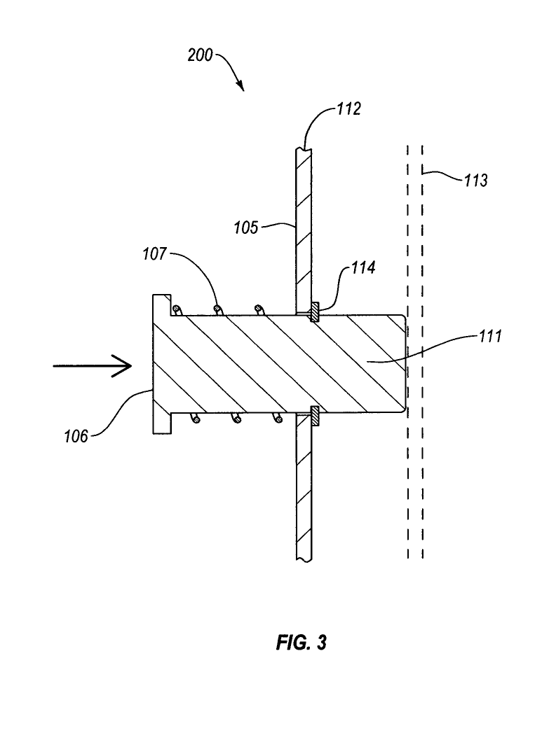

This rod may have a larger feature attached on its distal end such as a rectangular flat button face about 0.75 inches square and about 0.1875 inches thick, to create a surface on the button for the user to easily depress with a hand, finger or object. Further, a coil spring (107) is placed over the plastic rod between the surface of the User Side of the USER INTERFACE BOARD and the 0.75 inch square face of the button. Further, a keeper ring is secured to the plastic rod on the proximal side of the button (the portion of the button that protrudes beyond the Rear Side of the USER INTERFACE BOARD) such that the spring and button assembly is trapped in the opening in the USER INTERFACE BOARD with the spring and square button face on the User Side of the board and the securing ring on the other side (Rear Side) of the USER INTERFACE BOARD. As a result, the described button has forward motion from its resting position when depressed by the user, against the force of the spring. This pressing action by the user, on the User Side of the USER INTERFACE BOARD causes the proximal end of the button to extend beyond its resting position by the distance that the spring is depressed, and beyond the Rear Side of the USER INTERFACE BOARD. The length of extension of the BUTTON beyond the Rear Side of the USER INTERFACE BOARD may be controlled by the length of the BUTTON and the travel allowed by compression of the coil spring. (See FIG. 3)

The herein disclosed components of the improved user interface system for a visual-motor and neuro-cognitive reaction measuring and training device (VMTD) are arranged in a preferred configuration, as follows:

a) The USER INTERFACE BOARD is mounted on its movable stand (Fig. Element 103) and positioned about 40 inches from the PROJECTOR (or another distance appropriate for the focus length of the selected PROJECTOR) at a height where the general center of the Board is about at the eye level of the user. There are no electrical or radio frequency connections between the USER INTERFACE BOARD, its BUTTONS or any of the other components of the VMTD.
b) The PROJECTOR (Fig. Element 102) is located on the rear side (FIG. 105) of the USER INTERFACE BOARD, on the wall, a stand or preferably on a support that extends from and is attached to the movable stand (FIG. 103) on which the USER INTERFACE BOARD is mounted; and the PROJECTOR is connected by wire or wirelessly to the SYSTEM CONTROLLER. This attachment of the PROJECTOR mount to the USER INTERFACE BOARD's stand, insures that the image of the PROJECTOR remains properly located on and accurately aligned with the Rear Side of the USER INTERFACE BOARD when the USER INTERFACE BOARD is raised or lowered to accommodate the height of various users.
c) The INFRARED EMITTER (Fig. Element 104) is mounted on the USER INTERFACE BOARD or movable stand or other surface such that it is positioned to generate its broad infrared beam or curtain parallel to and close to (within about 4-6 cm) of the full width of the Rear Side of the USER INTERFACE BOARD (FIG. 109) and is powered from its own AC driven low voltage transformer.
d) INFRARED CAMERA (Fig. Element 101) is mounted adjacent to the PROJECTOR just above or below the PROJECTOR with its focus on the Rear Side of the USER INTERFACE BOARD. The INFRARED CAMERA is connected to the SYSTEM CONTROLLER.
e) SYSTEM CONTROLLER is located adjacent to the USER INTERFACR BOARD with access to electrical Power.
f) Height adjustment of the USER INTERFACE BOARD on its movable stand is accomplished by a simple electrical servo motor that is controlled by a manual ‘up/down’ switch located on or near the USER INTERFACE BOARD's stand. Upward/downward movement may also be accomplished by manually lifting or lowering the USER INTERFACE BOARD.

Operation of the Invention

A) The SYSTEM CONTROLLER causes the PROJECTOR to project an area of visible light onto the Rear Side of the generally opaque USER INTERFACE BOARD. This projected light image is directed at and selectively illuminates only the area of the BOARD where the proximal portion of a selected BUTTON is located, causing the portion of the BUTTON on the face side of the BOARD to also be illuminated via transmission of the projected light through the clear plastic of the BUTTON.

Further, the area immediately around and adjacent to a BUTTON may be of a clear or translucent material such that the projected light that is directed at a specific BUTTON will also illuminate this area of the USER INTERFACE BOARD around and close to the illuminated BUTTON.

Because the PROJECTOR is able to project virtually any color of visible light specified by the SYSTEM CONTROLLER, a BUTTON and the adjacent area of the USER INTERFACE BOARD can be illuminated in a variety of colors.

As an alternative option, the BUTTON may be fabricated of a material that does not transmit the projected light from the Rear Side of the BUTTON to the User Side of the BUTTON, thus not illuminating the BUTTON on the User Side of the BOARD, such that the target BUTTON to be depressed by the user is designated only by illumination of the area of the USER INTERFACE BOARD immediately around the target BUTTON and not by illumination of the BUTTON itself.

Of interest and separate from the above teaching for the illumination of a BUTTON and as previously described, the PROJECTOR can also separately project an image onto the Rear Side of the translucent screen in the general center of the USER INTERFACE BOARD (Element 101); this image is displayed on the front/User Side of the USER INTERFACE BOARD in a manner that is well understood and practiced by rear projection viewing systems.

A) The user responds to the illumination of a BUTTON by pressing the BUTTON, causing the proximal portion of the BUTTON to be advanced into the infrared beam curtain that is created by the INFRARED EMITTER on the Rear Side of the generally opaque USER INTERFACE BOARD; disrupting the IR beam curtain on the Rear Side of the USER INTERFACE BOARD by causing IR light waves to be reflected off of that portion of the BUTTON protruding into the IR curtain on the Rear Side of the BOARD.
B) The INFRARED CAMERA, positioned on the Rear Side of the opaque USER INTERFACE BOARD, captures the location and change in the IR image that is caused by the user depressing the illuminated BUTTON causing this user depressed BUTTON to enter into the IR Curtain, changing the IR image such that the INFRARED CAMERA detects this IR image change and location, communicating this change to the SYSTEM CONTROLLER in a manner similar to a computer ‘mouse click’, thus causing the SYSTEM CONTROLLER to change the image projected by the PROJECTOR, such that the BUTTON that was illuminated and pressed by the user is now no longer illuminated and another BUTTON is immediately illuminated. This process is repeated, with the SYSTEM CONTROLLER measuring and recording the user's reaction time and accuracy in responding to the illumination of BUTTONS, until the BUTTON illumination routine that is pre-programmed into the SYSTEM CONTROLLER is completed.
C) Note: Per the referenced Ubi Interactive system and others; upon set-up, the IR CAMERA registers reference points on the projected image on the Rear Side of the USER INTERFACE BOARD, such that input from the IR CAMERA sent to the SYSTEM CONTROLLER will identify the screen location of the above referenced ‘mouse click’ (e.g., location of the specific BUTTON that has caused disruption of the IR Curtain).

The SYSTEM CONTROLLER program also controls the changing light pattern(s) projected onto the Rear Side of the USER INTERFACE BOARD that are viewed by the user on the front of the BOARD. The SYSTEM CONTROLLER also records the user's speed and accuracy in responding to the range of visualmotor and cognitive challenges presented by illumination of the BUTTONS and user reaction to the information displayed on the screen (FIG. 108) of the USER INTERFACE BOARD.

Further and relative to the operation of this invention, a number of motion interaction systems are commercially available including the Ubi Interactive projected touch screen and Xbox® Kinect systems. Both of these systems incorporate the use of an IR camera sensing a reflected change in an IR beam of light resulting from the user touching a projected display and disrupting an IR beam.

While the invention herein disclosed makes use of certain elements common to the commercially available optical touch detection presentation systems, this invention teaches a method of operation that uniquely overcomes limitations of such previous systems that make all such previous optical touch detection systems completely unusable to achieve a useful, effective and commercially viable device to measure, record and train the user's visual-motor and neuro-cognitive human performance (VMTD), as described above.

As previously explained herein, a most desirable and useful VMTD requires the incorporation of raised and illuminated 3D mechanical buttons that require that the user depresses the button by direct, specific and accurate contact so as not to depress adjacent buttons and to further provide tactile feedback of successful button activation to the user. In addition, this contact with a target button must be accurately and reliably recorded and timed by the VMTD to allow such system to be useful in the evaluation, training and rehabilitation of the user.

The current motion interaction systems (those referenced above and others) were created and are successfully used to impart touch screen features to an image projected on a wall or screen to facilitate a presentation, generally to a large group of people. This is accomplished by emitting an infrared light (IR) beam across and close to the surface of the user's side of the screen image that is projected on the screen, such that when a user positioned in front of the projected image touches the screen, the user's finger or hand disrupts this IR beam and such disruption is captured by an IR camera that is positioned with the Projector on the user side (front) of the screen. While such systems are innovative and useful for their intended purpose, they are, as presently designed and used, wholly unsuited for use as a method for creating the described VMTD, for the following reasons;

A) In one version these systems project the image from in front of the screen or wall onto the face of the screen or wall. This results in shadows on the screen, cast by the user's presence between the projector and the screen, making the shadow areas non-active to touch activation. To minimize this problem the projector used in these systems is often mounted near the ceiling, above the screen and the user—projecting down at an angle onto the screen. While this is helpful in reducing the problem of shadows and can be a suitable option for this presentation touch screen application, it is not workable for the operation of a useful VMTD that requires that the user has complete and continuous view of the face of the entire board and all buttons, when the user is standing directly in front of the board. Further, this configuration, without mechanical buttons, would allow the user to simply swipe their hand across an area of the face of the screen in response to a changing visual event without providing for the recording of the accuracy and reaction time of the user's specific performance.
B) In another version, the current touch screen systems project the Image onto the rear of a translucent screen such that the image is seen by the user, positioned on the front side of the screen. This rear projection approach eliminates the issue of the user's visual shadows blocking the users unobstructed view of the projected image on the screen but does not address the blocking of portions of the screen (by the user's body) from being viewed by the IR Camera. With the IR Camera blocked, the system cannot register the user's touch of the screen in areas of the screen that are blocked from the view of the IR Camera. This is not a usable arrangement for a VMTD application.
C) In all embodiments of the optical touch detection systems that are commercially available, as described, the IR beam is projected on the user side of the screen. As is explained above, this requirement of these present systems where the user's touch interaction with the projected image can be ‘seen’ by the IR camera, makes these systems wholly unusable to support the operation of a useful and effective VMTD.
D) Unlike all currently available projected optical touch detection systems that track and respond to the user's touch of the screen in virtually any location on the image that is being projected, a VMTD, as described, must illuminate only at fixed locations, such as the specific location of the BUTTONS attached to the User Interface Board and the information screen located in the general center of the User Interface Board.

This invention, as herein described, successfully addresses and solves these limitations such that an optical touch detection system, as disclosed herein, can be created as the basis for the operation of an effective, useful and commercially viable User Interface System for a visual-motor and neuro-cognitive human performance (VMTD), in the following manner;

A) In addition to the rear projection of the light images onto the rear of the screen and onto the rear portion of the BUTTON by the projector, to be seen by the user on the front side of the screen, the IR Emitter, IR beam and IR camera are, in this teaching, located on the rear side of the screen (not the user side).
B) The screen is formed of a flat, ridged, board like structure that is generally opaque except for openings/holes from its front side through to its rear side and such holes are occupied by mechanical BUTTONS (not projected onto a fully reflective surface, visible to the user, as in currently available projected optical touch detection systems).
C) The IR beam emitted on the rear side of the opaque screen is caused to be disrupted by a portion of a BUTTON that is caused to enter the IR beam when such BUTTON is depressed by the user, who is located on the front side of the screen; opposite of the side of the board that is illuminated by the projector and the IR Emitter and addressed by the IR camera.
D) Illumination of only selected and fixed portions of the front surface of the screen and selected BUTTONS is the result of visible light projected on specific and fixed translucent portions of the rear surface of the largely opaque screen by the PROJECTOR.

### DESCRIPTION OF DRAWINGS

FIG. 1/3 (Sheet 100) Front/side view of the disclosed invention showing—
IR Projector (101); Visible Light Projector (102); Adjustable Stand (103); IR Emitter (104); Button, Face of User Interface Board (105); User side of Button (106); Spring on Button (107); Translucent window (108); User Interface Board (112); IR Light Curtain (113)

FIG. 2/3 (Sheet 100) Rear/side view of the disclosed Invention—
Rear side of User Interface Board (109); Rear Portion of Button (111); Retaining Ring (114)

FIG. 3/3 (Sheet 200); Side View of Button Assembly—
See A & B, above.

What is claimed:

1. A method for making and using a visual motor neuro-cognitive reaction training and evaluation device (VMTD) as previously herein described, wherein the user responds to the illumination of a 3 dimensional mechanical button that is mounted in a flat, opaque board, by depressing the said button and wherein the VMTD registers the user's response to the illumination of the button by the user pressing the illuminated button, and where there is no wired electrical connection or wireless radio frequency connection to or from the illuminated mechanical button, and where all such communication to and from the illuminated button and the illumination of the button is achieved by creating changes in the pattern of projected visible and infrared light energy detected by an infrared camera as previously described in the OPERATION OF THE INVENTION portion of this invention.

2. The method of claim 1 where there are a plurality of buttons mounted in a flat, opaque board that can be caused to illuminate in a planned or random order by the SYSTEM CONTROLLER.

3. The method of claim 1 where there are a plurality of buttons mounted in a flat, opaque board that can be caused to be illuminated in different colors by the SYSTEM CONTROLLER.

4. A method for making and using a visual motor reaction device wherein the depression of a 3 dimensional mechanical button by the user on the front side of the flat, generally opaque board causes disruption of an infrared light beam that is projected on the rear side of the flat, generally opaque board adjacent to the portion of the button on the rear surface of said board wherein the depression of the button by the user on the front surface of the board causes the button to disrupt the IR beam on the rear surface of the said board, and where such disruption is detected by an IR camera that is positioned to image the back side of the said board and the IR camera is in connection with the computer based operating system of the device, causing the device to recognize and respond to the user's depression of the button.

5. The teachings of claim 4 wherein the portion of the button that disrupts the infrared (IR) light beam when the button is depressed has an IR specific coating that increase the IR reflection or increases the IR absorption in that portion of the button that enters the IR light beam when the button is depressed, thereby creating a greater change in the IR image, than an uncoated button, when the button is depressed.

6. The teachings of claim 4 wherein the button that is depressed by the user causes by direct action, another adjacent object to disrupt the infrared (IR) light beam when the button is depressed.

7. A method for making and using a visual motor reaction device wherein the illumination of a 3 dimensional mechanical button mounted in an opaque board is achieved by the rear projection of light onto a portion of the button, where said button is comprised of a light transmitting material that transmits the rear projected illumination that is directed at the rear of the board, through such board to illuminate the portion of the button on the front surface of the opaque board.

8. The teachings of claim 7 wherein the BUTTON that is depressed by the user is opaque to light and is not illuminated by the PROJECTOR but is designated as the target light for the user to press by the illumination of the immediate area around such BUTTON where such areas are translucent.
